SEQUENCE LISTING (22 sequences)

SEQ ID NO: 1 - DNA, 19 bp, synthetic construct
  source 1..19: mol_type = other DNA
gggcctgcacctgctgcag

SEQ ID NO: 2 - DNA, 19 bp, synthetic construct
  source 1..19: mol_type = other DNA
gcacgtagtctgagtgctg

SEQ ID NO: 3 - DNA, 24 bp, synthetic construct
  source 1..24: mol_type = other DNA
ctcaggaggagcaatgatcttgat

SEQ ID NO: 4 - DNA, 20 bp, synthetic construct
  source 1..20: mol_type = other DNA
taccaccatgtacccaggca

SEQ ID NO: 5 - DNA, 18 bp, synthetic construct
  source 1..18: mol_type = other DNA
cctgagcccggaggagaa

SEQ ID NO: 6 - DNA, 18 bp, synthetic construct
  source 1..18: mol_type = other DNA
ctcgagcagtctgcgctg

SEQ ID NO: 7 - DNA, 20 bp, synthetic construct
  source 1..20: mol_type = other DNA
atcaggacgcgcaaacatga

SEQ ID NO: 8 - DNA, 19 bp, synthetic construct
  source 1..19: mol_type = other DNA
ttgagggatgatcgctgct

SEQ ID NO: 9 - DNA, 19 bp, synthetic construct
  source 1..19: mol_type = other DNA
cccagatgacttgtcccca

SEQ ID NO: 10 - DNA, 20 bp, synthetic construct
  source 1..20: mol_type = other DNA
ttgagggatgatcgctgctg

SEQ ID NO: 11 - DNA, 20 bp, synthetic construct
  source 1..20: mol_type = other DNA
aagaacacgcttgggaatgg

SEQ ID NO: 12 - DNA, 17 bp, synthetic construct
  source 1..17: mol_type = other DNA
ctgcacctgctgcggac

SEQ ID NO: 13 - DNA, 21 bp, synthetic construct
  source 1..21: mol_type = other DNA
aggaactggcaaaaggatggt

SEQ ID NO: 14 - DNA, 20 bp, synthetic construct
  source 1..20: mol_type = other DNA
atgttgttgctgatggcctg

SEQ ID NO: 15 - DNA, 20 bp, synthetic construct
  source 1..20: mol_type = other DNA
gcgagaagatgacccagatc

SEQ ID NO: 16 - DNA, 19 bp, synthetic construct
  source 1..19: mol_type = other DNA
ccagtggtacggccagagg

SEQ ID NO: 17 - DNA, 18 bp, synthetic construct
  source 1..18: mol_type = other DNA
ctgagtccgcagcaggtg

SEQ ID NO: 18 - DNA, 20 bp, synthetic construct
  source 1..20: mol_type = other DNA
tccaagttgtccagaatgcc

SEQ ID NO: 19 - DNA, 19 bp, synthetic construct
  source 1..19: mol_type = other DNA
cgccaaatcaagaaggcct

SEQ ID NO: 20 - DNA, 21 bp, synthetic construct
  source 1..21: mol_type = other DNA
cagcatccgggctcttatttt

SEQ ID NO: 21 - DNA, 21 bp, synthetic construct
  source 1..21: mol_type = other DNA
cttggaatctccaccgcaatg

SEQ ID NO: 22 - DNA, 20 bp, synthetic construct
  source 1..20: mol_type = other DNA
gtagtccacgttcccatcag

(19) United States
(12) Patent Application Publication
(10) Pub. No.: US 2026/0097077 A1
(43) Pub. Date: Apr. 9, 2026

(54) Methods and compositions for cancer therapy using modified gamma delta T cells

(71) Applicants: Research & Business Foundation SUNGKYUNKWAN UNIVERSITY, Suwon-si (KR); NATIONAL CANCER CENTER, Goyang-si (KR)

(72) Inventors: Minkyung SONG, Gwacheon-si (KR); Hocheol SHIN, Suwon-si (KR); Ji Yun PARK, Hwaseong-si (KR)

(21) Appl. No.: 19/318,631

(22) Filed: Sep. 4, 2025

(30) Foreign Application Priority Data
Oct. 7, 2024 (KR) 10-2024-0135756

(51) Int. Cl. A61K 35/17; A61P 35/00; C12N 9/22; C12N 15/11; C12N 15/86; C12N 15/90

(52) U.S. Cl. CPC A61K 35/17; A61P 35/00; C12N 9/226; C12N 15/11; C12N 15/86; C12N 15/907; C12N 2310/20; C12N 2740/15043

(57) ABSTRACT

The present invention relates to a method for treating cancer, comprising administering a composition comprising γδ T cells treated with an inhibitor of XBP1 gene expression or an inhibitor of XBP1 protein expression or activity. The γδ T cells, in which the XBP1 gene is deficient or the XBP1 protein activity is inhibited, exhibit enhanced antitumor activity in a tumor microenvironment characterized by endoplasmic reticulum (ER) stress, as compared to unmodified γδ T cells. Accordingly, the modified γδ T cells are effective for use in cancer treatment.

### CROSS-REFERENCE TO RELATED APPLICATION

This application is based on and claims priority from Korean Patent Application No. 10-2024-0135756 filed on Oct. 7, 2024 in the Korean Intellectual Property Office, the content of which is incorporated herein by reference in its entirety.

### REFERENCE TO AN ELECTRONIC SEQUENCE LISTING

The contents of the electronic sequence listing (JPD20250047US_SEQ.xml; Size: 19, 881 bytes; and Date of Creation: Dec. 17, 2025) is herein incorporated by reference in its entirety. The contents of the electronic sequence listing in no way introduces new matter into the specification.

### BACKGROUND

### Field of the Invention

The present invention relates to a method for treating cancer, comprising administering a composition comprising γδ T cells treated with an inhibitor of XBP1 gene expression or an inhibitor of XBP1 protein expression or activity to a subject in need thereof; and a method for treating cancer, comprising administering a first agent that inhibits XBP1 expression, and a second agent comprising γδ T cells, to the subject in need thereof.

### Related Art

Cancer is one of the leading causes of death worldwide, with approximately 20 million new cases and 10 million deaths reported in 2022. In Korea, the cancer-related mortality rate is 162.7 per 100,000 population, accounting for 22.4% of all deaths, making cancer the primary cause of death in both men and women. Conventional cancer treatments include surgery, radiotherapy, chemotherapy, and targeted therapies. While these treatments may provide short-term efficacy, they can cause severe side effects in patients and, over time, resistance may develop, leading to cancer recurrence and metastasis. Given the limited alternative options for patients with treatment resistance and the poor therapeutic responses, there is an urgent need for new, effective treatments.

Immunotherapeutic agents that have recently emerged activate or restore the patient's immune system, ultimately inducing durable immune anticancer responses through conventional αβ T cell-mediated cytotoxicity and immune memory formation. However, for solid tumors of epithelial origin, the use of such agents is limited, and reported response rates are often less than 20%. In solid tumors and metastatic cancers, the clinical efficacy of existing immunotherapies is further reduced by genetic heterogeneity within tumor tissues and by the loss of tumor antigens. In addition, multiple immunosuppressive factors in the tumor microenvironment diminish their effectiveness. In particular, the rapid proliferation and high metabolic activity of cancer cells, together with aberrant angiogenesis, create a tumor microenvironment characterized by hypoxia, oxidative stress, nutrient deprivation, and acidosis. Under these conditions, endoplasmic reticulum (ER) stress is induced in tumor-infiltrating immune cells, and subsequent activation of the signaling pathways mediated by ER stress sensors such as IRE1α and PERK suppresses anticancer immune function. Notably, T cell activity is inhibited when the IRE1α-XBP1 signaling pathway is persistently overactivated without restoration of normal ER function.

γδ T cells, which constitute approximately 5% of peripheral blood T cells, recognize and eliminate cancer cells through an MHC-independent mechanism, unlike conventional αβ T cells. In addition, γδ T cells promote broad anticancer immunity by inducing the activation of other immune cells. Although γδ T cell-based cytotoxicity has been reported to be effective against various cancers, their expected antitumor activity is often suppressed once adoptively transferred γδ T cells infiltrate tumors. Therefore, there remains a need for strategies that preserve the antitumor functions of immune cells within the hostile tumor microenvironment.

### SUMMARY OF THE INVENTION

The present invention is directed to a method for treating cancer in a subject in need thereof, the method comprising: administering a therapeutically effective amount of a composition comprising γδ T cells modified by an inhibitor of X-box binding protein 1 (XBP1) gene expression or an inhibitor of XBP1 protein expression or activity.

The present invention is directed to a method for treating cancer in a subject in need thereof, the method comprising administering to the subject a therapeutically effective amount of (a) a first agent comprising an inhibitor of XBP1 expression; and (b) a second agent comprising γδ T cells, wherein the combined administration of (a) and (b) enhances antitumor activity.

The present invention is directed to a method for manufacturing cell therapy composition for treating cancer, the method comprising (a) isolating γδ T cells from peripheral blood mononuclear cells (PBMCs); and (b) generating XBP1-deficient γδ T cells by inhibiting or deleting the XBP1 gene in the γδ T cells isolated in step (a).

The present invention is directed to a cell therapy composition for preventing or treating cancer, comprising γδ T cells in which XBP1 gene expression or protein expression or activity is inhibited by an active reagent.

The present invention is directed to a personalized kit for preventing or treating cancer, comprising the γδ T cell composition.

The present invention is directed to a pharmaceutical composition for combined administration, comprising (a) an agent that inhibits XBP1 expression, and (b) γδ T cells.

Accordingly, the present invention provides methods and compositions wherein γδ T cells in which XBP1 gene expression or XBP1 protein expression or activity is inhibited exhibit superior antitumor activity compared to wild-type γδ T cells in tumor microenvironments characterized by ER stress, thereby enabling more effective cancer treatment.

### BRIEF DESCRIPTION OF THE DRAWINGS

FIG. 1 illustrates changes in the antitumor activity of mouse-derived γδ T cells following induction of ER stress.

FIG. 2 illustrates the evaluation of antitumor activity of adoptively transferred XBP1-deficient γδ T cells in a mouse model of ovarian cancer.

FIG. 3 illustrates phenotypic changes in human γδ T cells following pharmacological inhibition of XBP1 under ER stress conditions.

FIG. 4 illustrates changes in the antitumor activity of human γδ T cells following pharmacological inhibition of XBP1 under ER stress conditions.

FIG. 5 illustrates the generation of human XBP1-deficient γδ T cells using a CRISPR/Cas9 system.

FIG. 6 illustrates the evaluation of antitumor activity of γδ T cells under ER stress in a cell-derived xenograft (CDX) mouse model.

### DETAILED DESCRIPTION OF THE EMBODIMENTS

Hereinafter, the present invention will be described in detail with reference to exemplary embodiments.

The terms used in the present invention are general terms commonly used in the art, selected in consideration of their functions in the present invention, but their meanings may vary depending on the intent of those skilled in the art, prior usage, or the advent of new technologies. Additionally, in certain cases, terms may be arbitrarily selected by the applicant, and in such instances, their meanings are defined in the relevant description of the present invention. Accordingly, the terms used herein should be interpreted based on their intended meaning and the overall context of the invention, rather than merely on their literal expression.

In the present invention, the term “comprising” with respect to any component or step does not exclude the presence of other components or steps, but may instead include the addition of such components or steps, unless otherwise specified.

The present invention provides a cell therapy composition for preventing or treating cancer, comprising yd T cells in which XBP1 gene expression or XBP1 protein expression or activity is inhibited, as an active ingredient.

As used herein, the term “T cell” refers to a lymphocyte that plays a role in antigen-specific adaptive immunity. T cells include naive T cells that have not yet encountered an antigen; and antigen-experienced mature T cells, which may differentiate into subsets such as helper T cells or cytotoxic T cells. In addition, memory T cells are generated after antigen exposure that persist to mediate long-term immune responses and include both αβ T cells and γδ T cells.

As used herein, the term “γδ T cells” refers to a small subset of T cells, comprising approximately 0.5 to 5% of human peripheral blood lymphocytes, that are functionally involved in both adaptive and innate immunity. Human γδ T cells are primarily divided into three major subsets based on their TCR δ chains, the Vδ1, Vδ2, and Vδ3. Among these, the majority of circulating γδ T cells express the Vδ2 chain paired with the Vγ9 chain. γδ T cells arise earlier in thymic development than αβ T cells, and display relatively limited TCR diversity. They can recognize non-peptide antigens, such as phosphoantigen-driven BTN molecules, and mount rapid immune responses, thereby functioning at the interface of innate and adaptive immunity.

As used herein, the term “cell therapy” refers to a therapeutic approach that involves the selection and expansion of viable autologous, allogeneic, or xenogeneic cells in vitro or modification of cellular characteristics by various methods, for the purpose of treatment, diagnosis, or prevention of disease, in order to restore or enhance cellular or tissue function. In regulatory practice, cell therapy has been classified as a drug since 1993 in the United States and since 2002 in Korea. Broadly, cell therapy can be classified into two categories: (a) stem cell therapy, aimed at tissue regeneration or restoration of organ function; and (b) immune cell therapy, aimed at modulating immune responses in vivo, including suppression or enhancement of immune activity.

As used herein, the term “XBP1 (X-box binding protein 1)” refers to a protein encoded by the XBP1 gene, which is a transcription factor that regulates the expression of genes important for proper functioning of the immune system and cellular stress response. Under endoplasmic reticulum stress, activation of ribonuclease (RNase) domain of IRE1α (inositol-requiring enzyme 1α) splices a 26 bp fragment from XBP1 mRNA. The spliced XBP1 mRNA is translated into XBP1s, which acts as a transcription factor promoting the expression of various chaperones to alleviate ER stress and restore ER homeostasis.

In the present invention, the inhibitor of XBP1 gene expression may be selected from the group consisting of microRNA (miRNA), small interfering RNA (siRNA), small hairpin RNA (shRNA), single guide RNA (sgRNA), guide RNA (gRNA), antisense oligonucleotides, and ribozymes, which specifically bind to the XBP1 gene, without limitation.

Preferably, the inhibitor of XBP1 gene expression may be an sgRNA designed based on the sequence of human XBP1 (X-box binding protein 1, NCBI accession Nos. NG_012266.1 and NM_005080.4). More preferably, the inhibitor of XBP1 gene expression may be an sgRNA comprising the nucleotide sequence of SEQ ID NO: 1 or SEQ ID NO: 2.

In the present invention, the inhibitor of XBP1 protein expression or activity may be selected from the group consisting of antibodies, aptamers, peptide nucleic acids (PNAS), peptides, peptidomimetics, targeted protein degraders (TPDs), and small molecule compounds, without limitation.

In particular, the inhibitor of XBP1 protein expression or activity may be a substance that binds to IRE1α and inhibits kinase or ribonuclease activity, thereby inhibiting splicing of XBP1 mRNA, or alternatively, a substance that directly binds to the XBP1 protein to inhibit its expression or activity.

Preferably, the inhibitor of XBP1 protein expression or activity may be MKC8866. MKC8866 is a pharmacological inhibitor targeting the ribonuclease domain of IRE1α and inhibits the splicing of XBP1 mRNA, thereby suppressing activation of downstream XBP1.

In the present invention, γδ T cells may be rendered functionally deficient in the XBP1 gene by an inhibitor of XBP1 gene expression, or may exhibit suppressed XBP1 protein expression or activity by an inhibitor of XBP1 protein expression or activity.

In the present invention, the γδ T cells may exhibit enhanced anticancer activity compared to wild-type γδ T cells, particularly in a tumor microenvironment in which endoplasmic reticulum stress is induced.

In the present invention, the cancer may be selected from the consisting of ovarian group cancer, cholangiocarcinoma, binary tract cancer, pancreatic cancer, breast cancer, kidney cancer, gastric cancer, lung cancer, liver cancer, colorectal cancer, skin cancer, bladder cancer, testicular cancer, uterine cancer, cervical cancer, thyroid cancer, brain tumor, head and neck cancer, prostate cancer, non-small cell lung cancer, colon cancer, squamous cell carcinoma, melanoma, myeloma, lymphoma, and leukemia, without limitation.

The cell therapy composition of the present invention may be administered through any conventional route by which it can reach the target tissue. For example, it may be administered intraperitoneally, intravenously, intramuscularly, subcutaneously, or intradermally, without limitation.

The composition may be formulated together with a pharmaceutically acceptable carrier generally used in cell therapy. The term “pharmaceutically acceptable” refers to a composition that is physiologically tolerable and does not generally cause adverse reactions such as gastrointestinal disorders, dizziness, or allergic reactions when administered to humans. Examples of pharmaceutically acceptable carriers include those for parenteral administration, such as water, suitable oils, saline, aqueous glucose, and glycols. The composition may further contain a stabilizer and a preservative. Suitable stabilizers include antioxidants such as sodium bisulfite, sodium sulfite, or ascorbic acid. Suitable preservatives include benzalkonium chloride, methyl- or propyl-paraben, and chlorobutanol. Additional examples of pharmaceutically acceptable carriers can be found in Remington's Pharmaceutical Sciences, 19^{th} ed., Mack Publishing Company, Easton, PA, 1995.

In addition, the composition may be administered using any device capable of delivering the cell therapy product to target cells.

The cell therapy composition of the present invention may comprise a therapeutically effective amount of the cell therapy product for treating a disease. The term “therapeutically effective amount” means an amount that induces a desired biological or medical response in a tissue system, animal, or human, as determined by researchers, veterinarians, physicians, or other clinicians. This includes an amount sufficient to relieve symptoms of the disease or disorder to be treated. It will be obvious to those skilled in the art that the cell therapy product contained in the composition of the present invention will be changed depending on the desired effect. Therefore, the optimal content of the cell therapy product may be determined by a person skilled in the art depending on factors such as the type and severity of the disease, the content of other components contained in the composition, the type of formulation, patient's age, body weight, health condition, sex, diet, administration route and timing, treatment period, secretion rate of the composition, and drugs used in combination with the composition. The cell therapy product should be included in the minimum amount that maximizes therapeutic effect while minimizing side effects, in view of all the above-described factors. For example, the composition of the present invention may contain 1×10⁴ cells/mL to 1×10⁸ cells/mL as the cell therapy agent.

The present invention also provides a personalized kit for preventing or treating cancer, comprising the cell therapy composition as an active ingredient.

In the present invention, γδ T cells may be isolated from peripheral blood mononuclear cells (PBMCs), which may be obtained from a subject in need of cancer treatment.

In the present invention, the γδ T cells obtained from a patient may be treated with an inhibitor of XBP1 gene expression or an inhibitor of XBP1 protein expression or activity, and then manufactured into a kit designed for re-administration or infusion into the patient.

In the present invention, the method for treating cancer or the composition for treating cancer may be suitable for personalized treatment.

The present invention also provides a pharmaceutical composition for combined administration for preventing or treating cancer, comprising a first agent comprising an inhibitor of XBP1 expression, and a second agent comprising γδ T cells.

In the present invention, the first agent and the second agent may be formulated separately and administered simultaneously or sequentially, without limitation.

The present invention also provides a method for treating cancer in a subject in need thereof, the method comprising administering a therapeutically effective amount of a composition comprising γδ T cells modified by an inhibitor of X-box binding protein 1 (XBP1) gene expression or an inhibitor of XBP1 protein expression or activity.

The present invention also provides a method for treating cancer in a subject in need thereof, the method comprising administering to the subject: (a) a first agent comprising an inhibitor of XBP1 expression; and (b) a second agent comprising γδ T cells, wherein the combined administration of (a) and (b) enhances antitumor activity.

Additional and redundant explanations are omitted hereinafter.

The present invention also provides a method for manufacturing a cell therapy composition for treating cancer, the method comprising: (a) expanding γδ T cells from peripheral blood mononuclear cells (PBMCs); and (b) generating XBP1-deficient γδ T cells by inhibiting or deleting the XBP1 gene in the γδ T cells obtained in step (a).

In the present invention, the XBP1-deficient γδ T cells may be generated using a CRISPR/Cas9 system, which may be delivered into the cells via a lentiviral vector, without limitation.

In the present invention, the XBP1 gene may be inhibited or deleted using a nucleic acid selected from the group consisting of small interfering RNA (siRNA), small hairpin RNA (shRNA), single guide RNA (sgRNA), guide RNA (gRNA), and antisense oligonucleotides. Preferably, the XBP1 gene may be inhibited or deleted using an sgRNA, more preferably an sgRNA comprising the nucleotide sequence of SEQ ID NO: 1 or SEQ ID NO: 2, without limitation.

In the present invention, the peripheral blood mononuclear cells may be isolated from a subject in need of cancer treatment.

In the present invention, the method may further comprise a step of expanding the XBP1-deficient γδ T cells ex vivo before formulating the cells into a pharmaceutical composition. Preferably, γδ T cells may be isolated from human peripheral blood mononuclear cells (PBMCs) after treatment with zoledronate and human recombinant IL-2, followed by selective ex vivo expansion, without limitation. Isolation of the XBP1-deficient γδ T cells may be performed using any conventional method for isolating γδ T cells from human peripheral blood mononuclear cells.

Hereinafter, the present invention will be further described through examples. These examples provide to illustrate the present invention in more detail, and the scope of the present invention is not limited thereto.

### Example 1. Changes in Antitumor Activity of Mouse-Derived γδ T Cells Under ER Stress

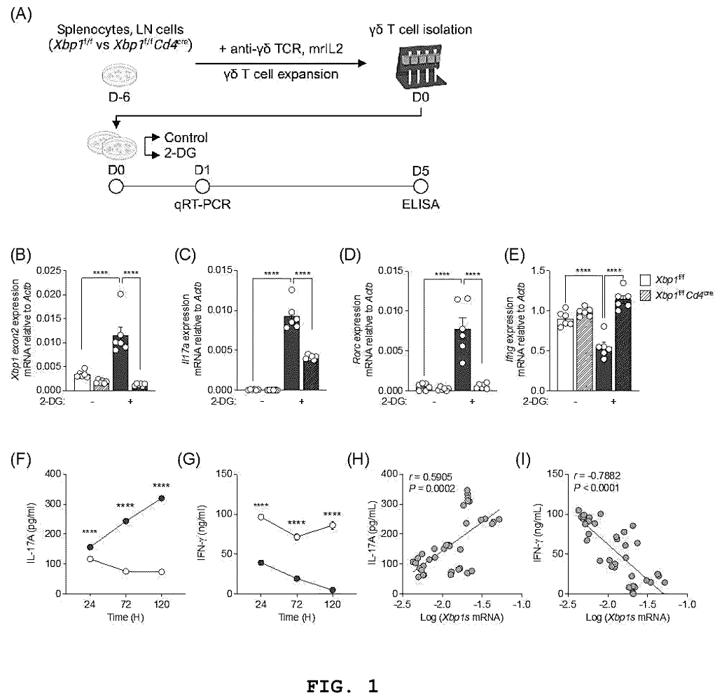

The XBP1-deficient mouse model was generated by crossing Xbp1^{f/f} mice with Cd4^{cre} mice to generate Xbp1^{f/f}Cd4^{cre} mice, in which XBP1 is selectively deleted in CD4-expressing cells. As shown in FIG. 1A, 8-week-old female Xbp1^{f/f} (WT) or Xbp1^{f/f}Cd4^{cre} (KO) mice were euthanized and sacrificed, and the spleens and lymph nodes were harvested. The tissues were homogenized using a cell strainer (SPL, 93070) and a syringe plunger, followed by treatment with ACK lysis buffer to remove red blood cells. The resulting cells were resuspended at a concentration of 3×10⁶ cells/mL and cultured on plates pre-coated with anti-γδTCR antibody (BioXCell). Cells were maintained in complete RPMI medium (CRPMI) supplemented with 10% heat-inactivated FBS (R&D Systems), 2 mM L-glutamine (Gibco), 1 mM sodium pyruvate (Gibco), 25 mM HEPES (Gibco), non-essential amino acids (Gibco), 100 IU/mL penicillin/streptomycin (Gibco), 55 μM beta-mercaptoethanol (Gibco), and 100 U/mL mouse recombinant IL-2 (Peprotech).

On day 6 of culture, γδ T cells were selectively isolated and then treated with 2 mM 2-DG (2-deoxy-D-glucose, Sigma Aldrich) for 24, 72, or 120 hours to induce ER stress. Following treatment, the cells were centrifuged at 1,500 rpm and then resuspended in QIAzol (Qiagen) for RNA extraction. The cell culture supernatant was collected and used to measure IL-17A and IFN-γ cytokine levels by ELISA.

Total RNA was isolated, and 300 ng of RNA was reverse transcribed into cDNA using the First Strand cDNA Synthesis Kit (Thermo Fisher Scientific) according to the manufacturer's instructions. Quantitative PCR was performed using PowerUP SYBR Green Master Mix and a QuantStudio™ 3 Real-Time PCR System (Applied Biosystems). Expression levels of the target genes Xbp1 exon2, Il17a, Rorc, Xbp1s, and Ifng, as listed in Table 1 below, were normalized to Actb as an internal control and analyzed by relative quantification.

TABLE 1

| | Forward/ | |
|---|---|---|
| Gene | Reverse | Sequence (5′ to 3′) |
| Actb | Forward | CTCAGGAGGAGCAATGATCTTGAT |
| | | (SEQ ID NO: 3) |
| | Reverse | TACCACCATGTACCCAGGCA |
| | | (SEQ ID NO: 4) |
| Xbp1 exon2 | Forward | CCTGAGCCCGGAGGAGAA |
| | | (SEQ ID NO: 5) |
| | Reverse | CTCGAGCAGTCTGCGCTG |
| | | (SEQ ID NO: 6) |
| Il17a | Forward | ATCAGGACGCGCAAACATGA |
| | | (SEQ ID NO: 7) |
| | Reverse | TTGAGGGATGATCGCTGCT |
| | | (SEQ ID NO: 8) |
| Rorc | Forward | CCCAGATGACTTGTCCCCA |
| | | (SEQ ID NO: 9) |
| | Reverse | TTGAGGGATGATCGCTGCTG |
| | | (SEQ ID NO: 10) |
| Xbp1s | Forward | AAGAACACGCTTGGGAATGG |
| | | (SEQ ID NO: 11) |
| | Reverse | CTGCACCTGCTGCGGAC |
| | | (SEQ ID NO: 12) |
| Ifng | Forward | AGGAACTGGCAAAAGGATGGT |
| | | (SEQ ID NO: 13) |
| | Reverse | ATGTTGTTGCTGATGGCCTG |
| | | (SEQ ID NO: 14) |

As a result, induction of ER stress increased the expression of Xbp1 exon2 mRNA in γδ T cells derived from Xbp1^{f/f} (WT) mice, whereas no such increase was observed in γδ T cells derived from Xbp1^{f/f}Cd4^{cre} (KO) mice (FIG. 1B). These findings confirm the efficient deletion of the Xbp1 exon 2 locus and the lack of functional XBP1 expression in Xbp1^{f/f}Cd4^{cre} (KO)-derived γδ T cells.

Furthermore, induction of ER stress increased the mRNA expression of Il17a and Rorc, which are indicative of a pro-tumor phenotype, in WT γδ T cells, while their expression was significantly reduced in KO γδ T cells (FIGS. 1C and 1D). In contrast, Ifng mRNA expression was reduced upon ER stress in WT γδ T cells but remained unchanged in KO γδ T cells (FIG. 1E).

In addition, IL-17A secretion progressively increased, whereas IFN-γ secretion decreased over time upon induction of ER stress in WT γδ T cells (FIGS. 1F and 1G). Correlation analysis revealed that IL-17A levels were positively correlated with the expression of spliced Xbp1 (Xbp1s), whereas IFN-γ levels were negatively correlated with the expression of Xbp1s (FIGS. 1H and 1I). Collectively, these results demonstrate that the XBP1 gene plays a regulatory role in modulating the expression of the pro-tumor cytokine IL-17A and the anti-tumor cytokine IFN-γ gene.

### Example 2. Evaluation of Antitumor Activity of Adoptively Transferred XBP1-Deficient γδ T Cells in a Mouse Model of Ovarian Cancer

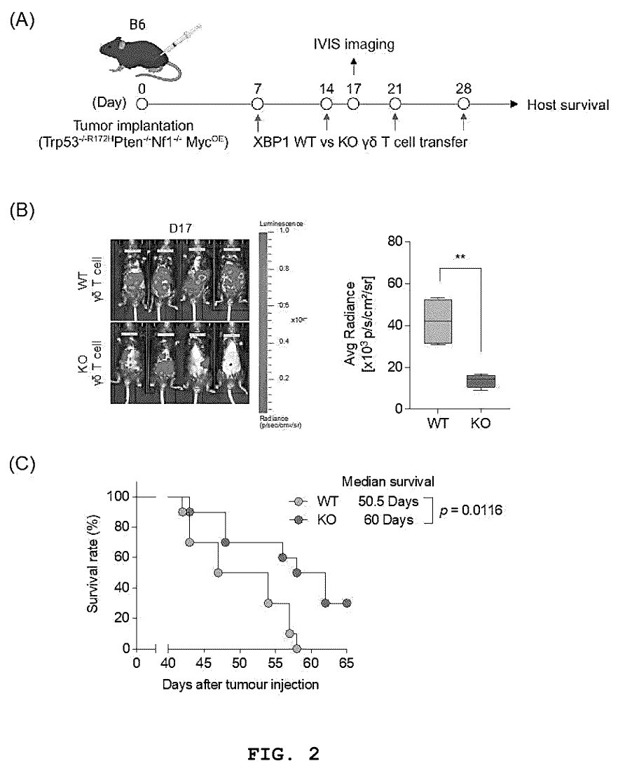

As shown in FIG. 2A, a syngeneic mouse model was established using the PPNM (Trp53^{-/-R172H}Pten^{-/-}Nf1^{-/-}Myc^{OE} genotype) cell line. Then, 1×10⁶ tumor cells were mixed with Matrigel (Corning) at a 1:1 ratio, and 100 μL of the mixture was injected intraperitoneally to C57BL/6 mice. Seven days after tumor cell implantation, γδ T cells derived from either XBP1 WT or KO mice were adoptively transferred intraperitoneally once a week (on day 7, day 14, day 21, and day 28). On day 17, 150 mg/kg VivoGlo™ luciferin (In Vivo Grade, Promega) was injected into the mice, and luminescence was assessed 20 minutes later using the Xenogen IVIS-100 imaging system (PerkinElmer). Images were analyzed with Living Image Software 4.8.0.

On day 17 after tumor implantation, a significant inhibition of tumor growth was observed in mice adoptively transferred with the XBP1-deficient γδ T cells compared with those transferred with WT γδ T cells (FIG. 2B). Furthermore, when survival was monitored up to 65 days post-tumor implantation, a significant increase in survival was observed in the mice transferred with XBP1-deficient γδ T cells compared with those transferred with WT γδ T cells (FIG. 2C).

These results confirmed that XBP1-deficient γδ T cells exert more potent anti-tumor effects than wild-type γδ T cells.

### Example 3. Phenotypic Changes in Human γδ T Cells Following Pharmacological Inhibition of XBP1 Under ER Stress Conditions

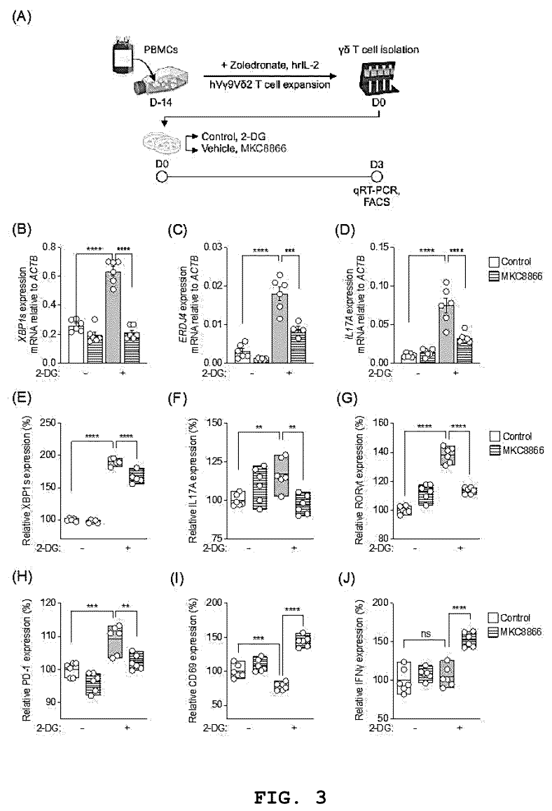

As shown in FIG. 3A, γδ T cells were expanded ex vivo by treating human PBMCs with zoledronate (Sigma, 1 μg/mL) and human recombinant IL-2 (Peprotech, 100 U/mL), followed isolation. The obtained γδ T cells were then cultured in CRPMI 1640 medium with 1 μM MKC8866 (MedChemExpress), a pharmacological inhibitor of IRE1α, at 37° C. in 5% CO₂ for 2 hours. Subsequently, the cells were treated with 5 mM 2-DG, an ER stress inducer, and cultured for 72 hours. After culture, the cells were centrifuged at 1,500 RPM for 5 minutes, and the precipitated cells were resuspended in QIAzol for RNA extraction or processed for flow cytometry analysis.

After RNA isolation, 300 ng of RNA was reverse transcribed into cDNA using the First Strand CDNA Synthesis Kit (Thermo Fisher Scientific) according to the manufacturer's instructions. Quantitative PCR was performed using the PowerUP SYBR Green Master Mix and QuantStudio™ 3 Real-Time PCR System (Applied Biosystems). The expression levels of the target genes XBP1s, ERDJ4 and IL17A as listed in Table 2 below were normalized to ACTB as an internal control and analyzed by relative quantification.

TABLE 2

| | Forward/ | |
|---|---|---|
| Gene | Reverse | Sequence (5′ to 3′) |
| ACTB | Forward | GCGAGAAGATGACCCAGATC |
| | | (SEQ ID NO: 15) |
| | Reverse | CCAGTGGTACGGCCAGAGG |
| | | (SEQ ID NO: 16) |
| XBP1s | Forward | CTGAGTCCGCAGCAGGTG |
| | | (SEQ ID NO: 17) |
| | Reverse | TCCAAGTTGTCCAGAATGCC |
| | | (SEQ ID NO: 18) |
| ERDJ4 | Forward | CGCCAAATCAAGAAGGCCT |
| | | (SEQ ID NO: 19) |
| | Reverse | CAGCATCCGGGCTCTTATTTT |
| | | (SEQ ID NO: 20) |
| IL17A | Forward | CTTGGAATCTCCACCGCAATG |
| | | (SEQ ID NO: 21) |
| | Reverse | GTAGTCCACGTTCCCATCAG |
| | | (SEQ ID NO: 22) |

As a result, the expression levels of ER stress-related genes, XBP1s and ERDJ4, were increased upon ER stress induction, whereas their expression was significantly reduced by pre-treatment with MKC8866 (FIGS. 3B and 3C). Similarly, the expression of the pro-tumorigenic cytokine IL17A was markedly upregulated under ER stress, but was effectively inhibited by MKC8866 pre-treatment (FIG. 3D).

For flow cytometry analysis, γδ T cells subjected to ER stress were centrifuged at 1,500 rpm for 5 min and stained with 100 μL of viability dye (BioLegend). FcRs were then blocked with TruStain (BioLegend), followed by staining with anti-XBP1s, anti-IL17A, anti-RORγt, anti-PD1, anti-CD69, and anti-IFNγ antibodies (all from BioLegend). The stained γδ T cells were analyzed using a BD FACS Canto2 (BD Biosciences).

Flow cytometry revealed that XBP1s protein expression was elevated under ER stress and inhibited by MKC8866 pre-treatment (FIG. 3E). Likewise, expression of IL17A and a transcription factor RORγt was increased under ER stress and suppressed upon MKC8866 treatment (FIGS. 3F and 3G).

Analysis of γδ T cell activity further showed that expression of PD-1, an exhaustion marker of T cells, increased under ER stress but decreased with MKC8866 pre-treatment (FIG. 3H). In contrast, expression of CD69, an activation marker, was reduced under ER stress but restored by MKC8866 (FIG. 3I). Moreover, expression of IFN-γ, an anti-tumor cytokine, was enhanced when γδ T cells were treated with MKC8866 during ER stress (FIG. 3J).

Taken together, these results demonstrate that ER stress derives γδ T cells toward a pro-tumor, IL17A-producing phenotype and promotes T cell exhaustion. However, treatment with MKC8866 preserved γδ T cell activation and anti-tumor cytokine production, thereby conferring a significant anti-tumor effect.

### Example 4. Changes in Antitumor Activity of Human γδ T Cells Following Pharmacological Inhibition of XBP1 Under ER Stress Conditions

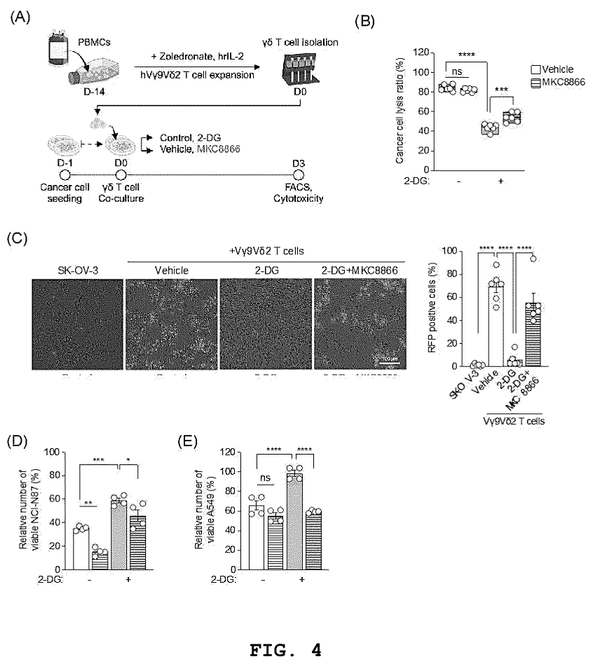

As shown in FIG. 4A, γδ T cells were selectively expanded ex vivo from PBMCs and subsequently isolated. The obtained γδ T cells were co-cultured with several cancer cell lines in cRPMI medium supplemented with 1 μM of MKC8866 at 37° C. and 5% CO₂ for 2 hours. Thereafter, the cells were treated with 5 mM of 2-DG, an ER stress inducer, and cultured under the same conditions for 72 hours. During culture, cells were monitored by live-cell imaging, and after completion of culture, subjected to flow cytometry analysis or cytotoxicity assay.

When the human ovarian cancer cell line SK-OV-3 was co-cultured with γδ T cells, cytotoxicity was assessed by flow cytometry. Results showed that under ER stress induced by 2-DG, the lysis rate of SK-OV-3 cells was reduced, leading to increased cancer cell viability (FIG. 4B). In contrast, pre-treatment with MKC8866 restored γδ T cell cytotoxicity, as evidenced by an increased lysis rate of SK-OV-3 cells (FIG. 4B).

To further analyze cancer cell death, real-time live-cell imaging was performed during co-culture of γδ T cells and cancer cells under ER stress. Cancer cells were pre-stained with the LIVE/DEAD viability/cytotoxicity kit (Invitrogen), and fluorescence (RFP) signals upon cell death were quantified. ER stress significantly reduced γδ T cell-mediated cancer cell death, whereas MKC8866 pre-treatment restored γδ T cell-mediated killing, resulting in increased cancer cell death (FIG. 4C). These results indicate that ER stress impairs, while MKC8866 restores, the anti-cancer function of γδ T cells.

Cytotoxicity was further evaluated using the CCK8 assay (Dojindo). The gastric cancer cell line NCI-N87 or lung cancer cell line A549 (1×10⁴ cells) were seeded in 96-well plates one day prior to co-culture with γδ T cells. The following day, γδ T cells were added at a 1:1 ratio with target cancer cells, and co-cultured for 72 hours at 37° C. and 5% CO₂. Cell viability was assessed with a Varioskan Lux plate reader (Thermo Fisher Scientific) according to the manufacturer's instructions. Results demonstrated that ER stress increased the viability of both cancer cell lines, while MKC8866 pre-treatment significantly enhanced the proportion of apoptotic cancer cells (FIGS. 4D and 4E).

Collectively, these findings confirm that MKC8866 pre-treatment restores the anti-cancer function of γδ T cells and rescues their ability to kill cancer cells, which was otherwise compromised by ER stress across multiple cancer types.

### Example 5. Generation of Human XBP1-Deficient γδ T Cells Using a CRISPR/Cas9 System

XBP1-deficient γδ T cells were generated by targeting the IRE1α cleavage region of XBP1 in human γδ T cells using a lentiviral CRISPR/Cas9 system. Lentivirus enables stable genomic integration of the editing machinery, ensuring heritable transmission of gene modifications upon cell division, and can efficiently infect both proliferating and resting cells. For these reasons, lentivirus was selected as the delivery vector for CRISPR/Cas9 into primary human γδ T cells.

Lentivirus was produced using HEK293T cells co-transfected with packaging and envelope plasmids, together with a transgene vector encoding sgRNA targeting XBP1 and eGFP-tagged Cas by using Lipofectamine 3000 (Invitrogen). Then, the cells were cultured under the conditions at 37° C. and 5% CO₂. The sgRNA sequences targeting XBP1 exon4 were SEQ ID NO: 1 (5′-GGGCCTGCACCTGCTGCAG-3′) or SEQ ID NO: 2 (5′-GCACGTAGTCTGAGTGCTG-3′). The culture supernatant containing virus particles was collected, clarified by centrifugation at 1,500 rpm at 4° C. for 5 minutes, passed through a 0.45-μm syringe filter, and concentrated with Retro-x concentrator (Takara) at a ratio of 3:1, and finally resuspended in T cell culture medium for transduction into γδ T cells.

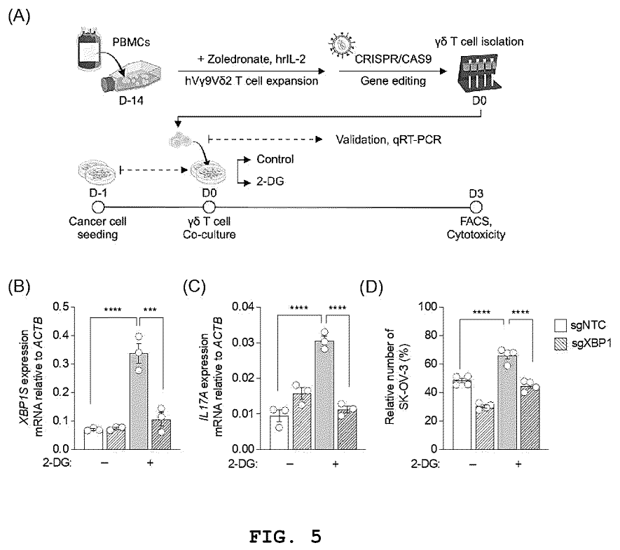

As shown in FIG. 5A, γδ T cells were selectively expanded ex vivo from PBMCs and then transduced with lentivirus at a volume ratio of 30%, four times at 12-hour intervals on days 4 and 5 of culture. After centrifugation at 1, 500 rpm for 10 minutes at room temperature, cells were maintained under 37° C. and 5% CO₂. On day 14, the transduced γδ T cells were harvested and subjected to co-culture experiments with cancer cell lines, followed by ER stress induction.

γδ T cells under ER stress were collected by centrifugation at 1,500 rpm and lysed in QIAzol for RNA extraction. cDNA synthesis was performed from 200 ng of RNA using the First Strand CDNA Synthesis Kit (Thermo Fisher Scientific). Quantitative PCR was performed with PowerUP SYBR Green Master Mix on a QuantStudio™ 3 Real-Time PCR System (Applied Biosystems). Expression levels of XBP1s and IL17A, as listed in Table 2 above, were normalized to ACTB as an internal control and analyzed by relative quantification.

Following lentiviral transduction of γδ T cells with CRISPR/Cas9 and XBP1-targeting sgRNA, cells were treated with 5 mM 2-DG for 72 hours. RNA analysis revealed that XBP1s expression increased in sgNTC-transduced cells upon 2-DG treatment, whereas sgXBP1-transduced cells maintained basal expression levels due to targeted gene editing (FIG. 5B). IL17A expression showed a similar pattern (FIG. 5C).

To assess functional consequences, SK-OV-3 cells were co-cultured with XBP1-deficient γδ T cells under ER stress. Wild-type γδ T cells exhibited impaired cytotoxicity under stress, whereas XBP1-deficient γδ T cells retained their ability to kill cancer cells (FIG. 5D).

Collectively, these results demonstrate that XBP1-deficient γδ T cells acquire resistance to ER stress and sustain their anti-cancer effector function.

### Example 6. Evaluation of Antitumor Activity of γδ T Cells Under ER Stress in a Cell-Derived Xenograft (CDX) Mouse Model

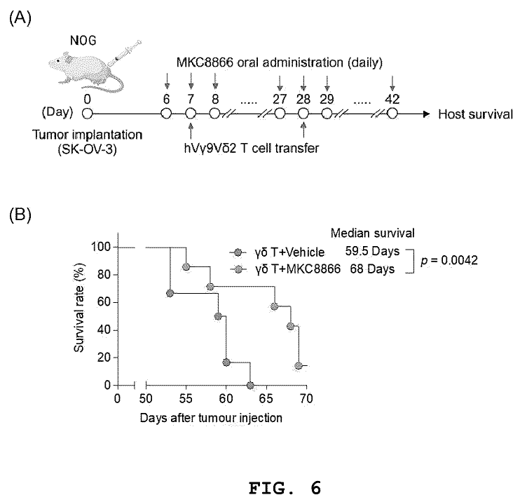

As shown in FIG. 6A, 1×10⁶ SK-OV-3 cells were suspended in Matrigel at a 1:1 ratio, and 100 μL of the mixture was injected intraperitoneally into immunodeficient mice. Beginning on day 6, MKC8866 was orally administered at 150 mg/kg daily until day 42. On days 7 and 28, 2×10⁶ human γδ T cells were adoptively transferred into the tumor-bearing mice.

As a result, the survival rate of the group receiving both γδ T cells and MKC8866 was significantly higher than that of the group receiving γδ T cells alone (FIG. 6B).

These findings indicate that combined administration of an XBP1 expression inhibitor with γδ T cells suppresses tumor growth and prolongs survival in tumor-bearing mice. Accordingly, combination therapy of XBP1 inhibition and γδ T cell immunotherapy may represent an effective therapeutic strategy.

The foregoing description of the present invention has been presented for illustrative purposes only, and those skilled in the art will recognize that the invention may be embodied in other specific forms without changing the technical idea or essential features of the present invention. Therefore, the embodiments describe above are to be considered exemplary in all respects and not restrictive. For example, each component described as integrated may be implemented separately, and likewise, components described as separate components may be implemented in an integrated form.

The scope of the present invention is defined by the claims described below, and all changes or modifications derived from the meaning and scope of the claims and the equivalent concepts thereof should be interpreted as being included in the scope of the present invention.

What is claimed is:

1. A method for treating cancer in a subject in need thereof, the method comprising:
administering a therapeutically effective amount of a composition comprising γδ T cells modified by an inhibitor of X-box binding protein 1 (XBP1) gene expression or an inhibitor of XBP1 protein expression or activity.

2. The method of claim 1, wherein the inhibitor of XBP1 gene expression is selected from the group consisting of microRNA (miRNA), small interfering RNA (siRNA), small hairpin RNA (shRNA), single guide RNA (sgRNA), guide RNA (gRNA), antisense oligonucleotides, and ribozymes, which specifically bind to the XBP1 gene.

3. The method of claim 1, wherein the inhibitor of XBP1 gene expression is a sgRNA comprising the nucleotide sequence of SEQ ID NO: 1 or SEQ ID NO: 2.

4. The method of claim 1, wherein the inhibitor of XBP1 protein expression or activity is selected from the group consisting of an antibody, an aptamer, a peptide nucleic acid (PNA), a peptide, a peptidomimetic, a targeted protein degrader (TPD), and a small molecule compound, which inhibits XBP1 protein expression or activity.

5. The method of claim 1, wherein the γδ T cells are deficient in the XBP1 gene.

6. The method of claim 1, wherein the γδ T cells reduce the XBP1 protein expression or activity.

7. The method of claim 1, wherein the γδ T cells exhibit enhanced anticancer activity relative to unmodified γδ T cells.

8. The method of claim 1, wherein the cancer is selected from the group consisting of ovarian cancer, cholangiocarcinoma, bile duct cancer, pancreatic cancer, breast cancer, kidney cancer, gastric cancer, lung cancer, liver cancer, colorectal cancer, skin cancer, bladder cancer, testicular cancer, uterine cancer, cervical cancer, thyroid cancer, brain tumor, head and neck cancer, prostate cancer, non-small cell lung cancer, colon cancer, squamous cell carcinoma, melanoma, myeloma, lymphoma, and leukemia.

9. The method of claim 1, wherein the γδ T cells are isolated from peripheral blood mononuclear cells (PBMCs).

10. The method of claim 9, wherein the peripheral blood mononuclear cells (PBMCs) are obtained from a subject in need of cancer treatment.

11. The method of claim 1, wherein the method is for personalized treatment.

12. A method for treating cancer in a subject in need thereof, the method comprising administering to the subject a therapeutically effective amount of:
(a) a first agent comprising an inhibitor of XBP1 expression; and
(b) a second agent comprising γδ T cells,
wherein the combined administration of (a) and (b) enhances antitumor activity.

13. The method of claim 12, wherein the first agent and the second agent are formulated separately and are administered simultaneously or sequentially.

14. A method for manufacturing cell therapy composition for treating cancer, the method comprising:
(a) isolating γδ T cells from peripheral blood mononuclear cells (PBMCs); and
(b) generating XBP1-deficient γδ T cells by inhibiting or deleting the XBP1 gene in the γδ T cells isolated in step (a).

15. The method of claim 14, wherein the XBP1-deficient γδ T cells are generated using a CRISPR/Cas9 system.

16. The method of claim 15, wherein the CRISPR/Cas9 system is delivered into the cells via lentivirus.

17. The method of claim 14, wherein the XBP1 gene is inhibited or deleted by using a nucleic acid selected from the group consisting of siRNA, shRNA, sgRNA, gRNA, and antisense oligonucleotides.

18. The method of claim 14, wherein the XBP1 gene is inhibited or deleted by using a sgRNA comprising the nucleotide sequence of SEQ ID NO: 1 or SEQ ID NO: 2.

19. The method of claim 14, wherein the peripheral blood mononuclear cells (PBMCs) are isolated from a subject in need of cancer treatment.

20. The method of claim 14, further comprising expanding the XBP1-deficient γδ T cells ex vivo before formulating them into a pharmaceutical composition.
